(19) United States
(12) Patent Application Publication
(10) Pub. No.: US 2024/0085431 A1
(43) Pub. Date: Mar. 14, 2024

(54) METHODS FOR PROVIDING AND DETERMINING AN EFFICACIOUS WEIGHT LOSS DIET FOR A CANINE

(71) Applicant: Société des Produits Nestlé S.A., Vevey (CH)

(72) Inventors: Yuanlong Pan, Chesterfield, MO (US); Hui Xu, Chesterfield, MO (US); Sandeep Bhatnagar, Ballwin, MO (US); Julie Kristine Spears, St. Louis, MO (US)

(21) Appl. No.: 18/366,277

(22) Filed: Aug. 7, 2023

Related U.S. Application Data
(60) Provisional application No. 63/406,172, filed on Sep. 13, 2022.

(51) Int. Cl. G01N 33/68; A23K 50/40

(52) U.S. Cl. CPC G01N 33/6812; A23K 50/40; G01N 2800/52

(57) ABSTRACT

The present disclosure provides a method of determining the efficacy of a weight loss diet for a canine, the method comprising analyzing a serum or plasma sample from the canine for a biomarker before and after administration of the weight loss diet, wherein the biomarker is selected from the group consisting of 3-methylhistidine, 1-methylhistidine, and a combination thereof, and determining the weight loss diet is efficacious if the amount of the biomarker has decreased.

### CROSS REFERENCE TO RELATED APPLICATION

This application claims priority to U.S. Provisional Application Ser. No. 63/406,172 filed Sep. 13, 2022 the disclosure of which is incorporated in its entirety herein by this reference.

### BACKGROUND

Obese and overweight animals have an increased risk of many chronic diseases including heart disease, diabetes, hypertension, stroke, dyslipidemia, certain types of cancer, apnea and osteoarthritis. Therefore, it is essential for overweight and obese animals, including humans and pets, to lose excessive body fat to maintain health and quality of life. Unfortunately, losing excessive body fat or maintaining healthy weight after weight loss is difficult to achieve and various solutions can have adverse consequences, e.g., loss of lean body mass during weight loss or weight rebound after weight loss.

Obesity is among the most serious health problems in humans and pets and considered to be the leading preventable cause of death. Maintaining a healthy weight is critical for optimal metabolism, normal physical activity and good health. There is, therefore, a need for methods and compositions to increase satiety, promote body fat loss, and maintain lean body mass and healthy weight, for better the health and wellness of animals and methods to determine the efficacy of weight loss diets.

### SUMMARY

In one embodiment, a method of determining the efficacy of a weight loss diet for a canine can comprise analyzing a first serum or plasma sample from the canine for a biomarker selected from the group consisting of 3-methylhistidine, 1-methylhistidine, and a combination thereof; determining the amount of the biomarker in the first serum or plasma sample; analyzing a second serum or plasma sample for the biomarker after the canine has been administered the weight loss diet; determining the amount of the biomarker in the second serum or plasma sample; and determining the weight loss diet is efficacious if the amount of the biomarker in the second serum or plasma sample has decreased from the amount of the biomarker in the first serum or plasma sample or determining the weight loss diet is inefficacious if the amount of the biomarker in the second serum or plasma sample has not changed or increased from the amount of the biomarker in the first serum or plasma sample.

In another embodiment, a method of providing an efficacious weight loss diet for a canine can comprise analyzing a first serum or plasma sample from the canine for a biomarker selected from the group consisting of 3-methylhistidine, 1-methylhistidine, and a combination thereof; determining the amount of the biomarker in the first serum or plasma sample; analyzing a second serum or plasma sample for the biomarker after the canine has been administered the weight loss diet; determining the amount of the biomarker in the second serum or plasma sample; determining the weight loss diet is efficacious if the amount of the biomarker in the second serum or plasma sample has decreased from the amount of the biomarker in the first serum or plasma sample or determining the weight loss diet is inefficacious if the amount of the biomarker in the second serum or plasma sample has not changed or increased from the amount of the biomarker in the first serum or plasma sample; and wherein, when the weight loss diet is inefficacious, providing an efficacious weight loss diet to the canine that comprises protein, fat, carbohydrates, omega-3 fatty acids, and isoflavones; wherein the protein to carbohydrate is in a ratio ranging from 4.5:1 to 2.5:1 by weight as fed.

Other and further objects, features, and advantages of the invention will be readily apparent to those skilled in the art.

### DETAILED DESCRIPTION

### Definitions

The terms “treating”, “treat”, and “treatment” embrace both preventative, i.e., prophylactic, and palliative treatment.

The term “efficacious”, when referring to a weight loss diet, refers to a weight loss diet that maximizes loss of body fat and/or minimizes or prevents loss of lean body mass during administration of the weight loss diet to a canine.

The term “food” or “food product” or “food composition” means a product or composition that is intended for ingestion by a canine and provides nutrition to the canine.

The term “carbohydrate” refers to carbohydrates that are digestible, e.g., sugars and starches, and does not include fiber, e.g., cellulose or fermentable fibers.

The term “crude fiber” refers to part of insoluble fiber found in the edible portion of the plant cell wall, and crude fiber is a measure of the quantity of indigestible cellulose, lignin, and other components of this type in foods.

The term “total dietary fiber” refers to the portion of plant-derived food that cannot be completely broken down by canine digestive enzymes and includes both soluble and insoluble fibers. Soluble fiber dissolves in water and is fermented in the colon by gut microbiota. Examples of soluble fibers are beta-glucans, guar gum, Psyllium, inulin, wheat dextrin, resistant starches. Insoluble fiber does not dissolve in water. Examples of insoluble fibers are cellulose and lignin.

The term “regular basis” means at least monthly administration and, in one aspect, at least weekly administration. More frequent administration or consumption, such as twice or three times weekly, can be performed in certain embodiments. In one aspect, an administration regimen can comprise at least once daily consumption.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of the food compositions, or components thereof, physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The term “about” means plus or minus 20% of a numeric value; in one aspect, plus or minus 10%; in another aspect, plus or minus 5%; and in one specific aspect, plus or minus 2%. For example, in one aspect where about is plus or minus 20% of a numeric value, the phrase “from about 10% to about 20%” could include a range from 8% to 24% or 12% to 16%, include any subranges therein.

As used herein, embodiments, aspects, and examples using “comprising” language or other open-ended language can be substituted with “consisting essentially of” and “consisting of” embodiments.

The term “complete and balanced” when referring to a food composition means a food composition that contains all known required nutrients in appropriate amounts and proportions based on recommendations of recognized authorities in the field of canine nutrition and are therefore capable of serving as a sole source of dietary intake to maintain life or promote production, without the addition of supplemental nutritional sources. Nutritionally balanced pet food compositions are widely known and widely used in the art, e.g., complete and balanced food compositions formulated according to standards established by the Association of American Feed Control Officials (AAFCO). In one embodiment, “complete and balanced” can be according to the current standards published by AAFCO as of Jan. 1, 2022.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise, the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### DETAILED DESCRIPTION

The present methods are based upon the discovery that specific biomarkers can be used to determine the efficacy of weight loss diets.

In one embodiment, a method of determining the efficacy of a weight loss diet for a canine can comprise analyzing a first serum or plasma sample from the canine for a biomarker selected from the group consisting of 3-methylhistidine, 1-methylhistidine, and a combination thereof; determining the amount of the biomarker in the first serum or plasma sample; analyzing a second serum or plasma sample for the biomarker after the canine has been administered the weight loss diet; determining the amount of the biomarker in the second serum or plasma sample; and determining the weight loss diet is efficacious if the amount of the biomarker in the second serum or plasma sample has decreased from the amount of the biomarker in the first serum sample or determining the weight loss diet is inefficacious if the amount of the biomarker in the second serum or plasma sample has not changed or increased from the amount of the biomarker in the first serum or plasma sample.

In another embodiment, a method of providing an efficacious weight loss diet for a canine can comprise analyzing a first serum or plasma sample from the canine for a biomarker selected from the group consisting of 3-methylhistidine, 1-methylhistidine, and a combination thereof; determining the amount of the biomarker in the first serum sample; analyzing a second serum or plasma sample for the biomarker after the canine has been administered the weight loss diet; determining the amount of the biomarker in the second serum or plasma sample; determining the weight loss diet is efficacious if the amount of the biomarker in the second serum or plasma sample has decreased from the amount of the biomarker in the first serum or plasma sample or determining the weight loss diet is inefficacious if the amount of the biomarker in the second serum or plasma sample has not changed or increased from the amount of the biomarker in the first serum or plasma sample; and wherein, when the weight loss diet is inefficacious, providing an efficacious weight loss diet to the canine that comprises protein, fat, carbohydrates, omega-3 fatty acids, and isoflavones; wherein the protein to carbohydrate is in a ratio ranging from 4.5:1 to 2.5:1 by weight as fed.

Generally, the diet can be administered on a regular basis. In one embodiment, the weight loss diet can be administered to the canine for at least a month. In other aspects, the weight loss diet can be administered for at least 2 months, 3 months, 4 months, 5 months, 6 months, or even a year. The methods can further comprise administering the weight loss diet to the canine.

Additionally, the present methods can also comprise advising a different weight loss diet for the canine when the administered weight loss diet have been determined to be inefficacious. Generally, the advised weight loss diet is an efficacious weight loss diet. Such efficacious weight loss diets are further described herein.

Such efficacious weight loss diets generally have high protein and low carbohydrates, but also have a unique macronutrient profile, i.e., having specific ratios and components that provide specific health benefits. Notably, the present efficacious diets are not ketogenic diets (traditional or modified), i.e., diets that rely on high fat or diets having fat as the predominant component of the diet. Further, the present efficacious diet is set apart from general high protein diets as known in the art. Rather than relying on a single macronutrient component or ratio, the present methods and compositions rely on unique combination of macronutrient ratios and food components.

Generally, the present efficacious compositions comprise a protein. The protein can be crude protein material and may comprise vegetable proteins such as soybean meal, soy protein concentrate, corn gluten meal, wheat gluten, cottonseed, pea protein, canola meal, and peanut meal, or animal proteins such as casein, albumin, and meat protein. Examples of meat protein useful herein include beef, pork, lamb, equine, poultry, fish, and mixtures thereof. The compositions may also optionally comprise other materials such as dried whey and other dairy by-products. In one embodiment, the food compositions can comprise protein in amounts from about 30%, 35%, 40%, 45%, 50%, or even 55% to about 35%, 40%, 45%, 50%, 55%, or even 60% by weight, including various subranges within these amounts. In one aspect, the protein can be from about 40% to about 60% of the food composition by weight. In another aspect, the protein can be from about 45% to about 55% of the food composition by weight.

Notwithstanding the aforementioned proteins, the present efficacious compositions comprise isoflavones. In various embodiments, the isoflavones include at least one of daidzein, 6-O-malonyl daidzein, 6-O-acetyl daidzein, genistein, 6-O-malonyl genistein, 6-O-acetyl genistein, glycitein, 6-O-malonyl glycitein, 6-O-acetyl glycitein, biochanin A, or formononetin. The isoflavones or metabolites thereof can be from soybean (Glycine max) in certain embodiments. Where present, the one or more metabolites preferably include equol. In one embodiment, the food compositions can comprise isoflavones in amounts from about 300 mg, 400 mg, 500 mg, 600 mg, 700 mg, 800 mg, 900 mg, or even 1,000 mg per kg of the food composition to about 500 mg; 600 mg; 700 mg; 800 mg; 900 mg; 1,000 mg; 1,100 mg; 1,200 mg; 1,300 mg; 1,400 mg; or even 1,500 mg per kg of the food composition, including various subranges within these amounts. In one aspect, the isoflavones can present in an amount from about 100 mg to 1,500 mg per kilogram of the pet food composition. In another aspect, the isoflavones can present in an amount from about 300 mg to 1,200 mg per kilogram of the pet food composition.

Generally, any type of carbohydrate can be used in the efficacious food compositions. Examples of suitable carbohydrates include grains or cereals such as rⁱce, corn, millet, sorghum, alfalfa, barley, soybeans, canola, oats, wheat, rye, triticale and mixtures thereof. In one embodiment, the carbohydrate comprises from about 15% to about 25% of the food composition by weight. In another embodiment, the carbohydrate comprises from about 10% to about 20% of the food compositions by weight. In other aspects, the carbohydrate can be present in amounts from about 5%, 10%, 15%, or even 20%, to about 10%, 15%, 20%, or even 25% by weight.

Generally, the protein and carbohydrates are in ratios that provide a health benefit to the canine. Typically, the ratio of protein to carbohydrate ranges from 4.5:1 to 2.5:1 by weight. In some aspects, the ratio of protein to carbohydrate can range from 4.25:1 to 3.75:1, or even from 4.15:1 to 3:1 by weight.

Generally, the efficacious food compositions include fat. Examples of suitable fats include animal fats and vegetable fats. In one aspect, the fat source can be an animal fat source such as tallow, lard, or poultry fat. Vegetable oils such as corn oil, sunflower oil, safflower oil, grape seed oil, soybean oil, olive oil, fish oil and other oils rich in monounsaturated and n-6 and n-3 polyunsaturated fatty acids, may also be used. In one embodiment, the food compositions can comprise fat in amounts from about 10%, 15%, 20%, 25%, 30%, 35%, or even 40% to about 20%, 25%, 30%, 35%, 40%, or even 45%, including various subranges within these amounts by weight. In one aspect, the fat comprises from about 10% to about 40% of the food composition by weight. In another aspect, the fat comprises from about 10% to about 30% of the food composition by weight. In still another aspect, the fat comprises from about 10% to about 15% of the food composition by weight.

Notwithstanding the aforementioned fats, the present efficacious compositions comprise omega-3 fatty acids. Non-limiting examples of suitable omega-3 fatty acids include eicosapentaenoic acid (EPA), docosahexaenoic acid (DHA), alpha-linolenic acid (ALA), stearidonic acid (SDA), eicosatrienoic acid (ETE), eicosatetraenoic acid (ETA), heneicosapentaenoic acid (HPA), docosapentaenoic acid (DPA), tetracosapentaenoic acid, tetracosahexaenoic acid (nisinic acid) and mixtures thereof. In one embodiment, the omega-3 fatty acids can range from about 0.1%, 0.2%, 0.5%, 1%, 1.5%, 2%, 2.5%, or even 3% to about 1%, 1.5%, 2%, 2.5%, 3%, 3.5%, 4%, 4.5%, or even 5% of the composition by weight. In some embodiments, the omega-3 fatty acids are present in the food composition in an amount from about 0.1% to about 5% by weight. In some embodiments, the omega-3 fatty acids are present in the food composition in an amount from about 0.5% to about 2.5% by weight.

In addition to the fats and fatty acids discussed herein, the present efficacious compositions can comprise omega-6 fatty acids. Non-limiting examples of Ref No. 18931 suitable omega-6 fatty acids include linoleic acid (LA), gamma-linolenic acid (GLA), arachidonic acid (AA, ARA), eicosadienoic acid, calendic acid, dihomo-gamma-linolenic acid (DGLA), docosadienoic acid, adrenic acid, osbond acid, tetracosatetraenoic acid, tetracosapentaenoic acid, and mixtures thereof. In one embodiment, the omega-6 fatty acids can range from about 0.2%, 0.5%, 1%, 2%, or even 3% to about 1%, 2%, 3%, 4%, or even 5% of the composition by weight. In some embodiments, the omega-6 fatty acids are present in the food composition in an amount from about 1% to about 5% by weight. In some embodiments, the omega-6 fatty acids are present in the food composition in an amount from about 1% to about 2% by weight.

The administration can be performed on as-needed basis, an as-desired basis, a regular basis, or intermittent basis. In one aspect, the food composition can be administered to the canine on a regular basis. In one aspect, at least weekly administration can be performed. More frequent administration or consumption, such as twice or three times weekly, can be performed in certain embodiments. In one aspect, an administration regimen can comprise at least once daily consumption.

According to the presently described methods, administration, including administration as part of a dietary regimen, can span a period ranging from parturition through the adult life of the canine. In certain embodiments, the canine can be a young or growing canine. In other embodiments, administration can begin, for example, on a regular or extended regular basis, when the canine has reached more than about 10%, 20%, 30%, 40%, or 50% of its projected or anticipated lifespan. In some embodiments, the canine can have attained 40, 45, or 50% of its anticipated lifespan. In yet other embodiments, the canine can be older having reached 60, 66, 70, 75, or 80% of its likely lifespan. A determination of lifespan may be based on species, gender, size, genetic factors, environmental factors and stressors, present and past health status, past and present nutritional status, stressors, and the like.

Such administration can be performed for a time required to accomplish one or more objectives described herein, e.g., preserving lean body mass in a canine during weight loss. Other administration amounts may be appropriate and can be determined based on the canine's initial weight as well as other variables such as species, gender, breed, age, desired health benefit, etc.

The moisture content for such efficacious food compositions varies depending on the nature of the food composition. The efficacious food compositions may Ref No. 18931 be dry compositions (e.g., kibble), semi-moist compositions, wet compositions, or any mixture thereof. In one embodiment, the pet food composition can be a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one embodiment, the compositions can have moisture content from about 5% to about 20%. Dry food products include a variety of foods of various moisture contents, such that they are relatively shelf-stable and resistant to microbial or fungal deterioration or contamination. Also, in one aspect, dry food compositions can be extruded food products.

The efficacious food compositions may also comprise one or more fiber sources. Such fiber sources include fiber that is soluble, insoluble, fermentable, and nonfermentable. Such fibers can be from plant sources such as marine plants, but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, corn bran, wheat bran, oat bran, carob bean gum, citrus pulp, pectin, fructooligosaccharide, short chain oligofructose, mannanoligofructose, soy fiber, arabinogalactan, galactooligosaccharide, arabinoxylan, cellulose, chicory, or mixtures thereof.

Alternatively, the fiber source can be a fermentable fiber. Fermentable fiber has previously been described to provide a benefit to the immune system of a canine. Fermentable fiber or other compositions known to skilled artisans that provide a prebiotic to enhance the growth of probiotics within the intestine may also be incorporated into the composition to aid in the enhancement of the benefits described herein or to the immune system of a canine.

In one embodiment, the efficacious food compositions can include a total dietary fiber from about 1% to about 15% by weight. In some aspects, the total dietary fiber can be included in an amount from about 5% to about 15% by weight, or even from about 8% to about 13% by weight. In another embodiment, the food compositions can include crude fiber from about 1% to about 10% by weight. In some aspects, the crude fiber can be included in an amount from about 3% to about 10% by weight, or even from about 3% to about 7% by weight.

In some embodiments, the ash content of the food composition ranges from less than 1% to about 15%. In one aspect, the ash content can be from about 5% to about 10%.

Generally, the efficacious food composition can be suitable for consumption by a canine as a meal, component of a meal, a snack, or a treat. Such compositions can include complete foods intended to supply the necessary dietary requirements for a canine. In one embodiment, the diet can comprise a complete and balanced pet food. Examples of food compositions include but are not limited to dry foods, wet foods, drinks, bars, frozen prepared foods, shelf prepared foods, and refrigerated prepared foods.

The efficacious food compositions may further comprise one or more substances such as vitamins, minerals, antioxidants, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat-soluble vitamins as A, D, E, and K and water-soluble vitamins including B vitamins, and vitamin C. Inulin, amino acids, enzymes, coenzymes, and the like may be useful to include in various embodiments.

The present methods can provide a health benefit to the canine. In one embodiment, the health benefit can include preservation of lean body mass, minimization of lean body mass during weight loss, reduced body fat, reduced weight, reduced weight gain, reduced insulin resistance, decreased risk of diabetes, decreased risk of prediabetes, lower cholesterol, lower glucose, lower triglycerides, lower insulin, improved insulin sensitivity, lower leptin, prevention of prediabetes, delaying onset of prediabetes, treatment of prediabetes, prevention of diabetes, delaying onset of diabetes, treatment of diabetes, prevention of insulin resistance, delaying onset of insulin resistance, treatment of insulin resistance, prevention of overweight or obesity, delaying onset of overweight or obesity, treatment of overweight or obesity, promoting metabolic health, promoting better blood glucose management, lowering chronic inflammation and proinflammatory cytokines, improving voluntary daytime activity, reducing restlessness at daytime and nighttime, increasing satiety, and combinations thereof.

In various embodiments, the efficacious food compositions contain at least one of (1) one or more probiotics; (2) one or more inactivated probiotics; (3) one or more components of inactivated probiotics that promote health benefits similar to or the same as the probiotics, e.g., proteins, lipids, glycoproteins, and the like; (4) one or more prebiotics; and (5) combinations thereof. The probiotics or their components can be integrated into the food compositions (e.g., uniformly or non-uniformly distributed in the compositions) or applied to the food compositions (e.g., topically applied with or without a carrier). Such methods are known to skilled artisans, e.g., U.S. Pat. No. 5,968,569 and related patents.

Typical probiotics include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain can be selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g., NCC2627), Bifidobacterium sp. NCC2657 or Enterococcus faecium SF68 (NCIMB 10415). Generally, the food compositions can contain probiotics in amounts sufficient to supply from about 104 to about 10¹² cfu/canine/day, in one aspect, from 105 to about 10¹¹ cfu/canine/day, and in one specific aspect, from 107 to 10¹⁰ cfu/canine/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. Pat. No. 6,929,793. In one embodiment, the probiotic can be Enterococcus faecium SF68 (NCIMB 10415). In another embodiment, the probiotics can be encapsulated in a carrier using methods and materials known to skilled artisans.

As stated, the efficacious food compositions may contain one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, and isomaltulose. In one embodiment, the prebiotic can be chicory root, chicory root extract, inulin, or combinations thereof. Generally, prebiotics can be administered in amounts sufficient to positively stimulate the healthy microflora in the gut and cause these “good” bacteria to reproduce. Typical amounts range from about one to about 10 grams per serving or from about 5% to about 40% of the recommended daily dietary fiber for a canine. The probiotics and prebiotics can be made part of the composition by any suitable means. Generally, the agents can be mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, in one aspect, from about 0.3 to about 7%, and in one specific aspect, from about 0.5 to 5%, on a dry matter basis. The prebiotics can be integrated into the compositions using methods known to skilled artisans, e.g., U.S. Pat. No. 5,952,033.

A skilled artisan can determine the appropriate amount of food ingredients, vitamins, minerals, probiotics, prebiotics, antioxidants, or other ingredients to be used to make a particular composition to be administered to a particular canine. Such artisan can consider the canine's species, age, size, weight, health, and the like in determining how best to formulate a particular composition comprising such ingredients. Other factors that may be considered include the desired dosage of each component, the average consumption of specific types of compositions by different canine (e.g., based on species, body weight, activity/energy demands, and the like), and the manufacturing requirements for the composition.

In a further aspect, the present disclosure provides kits suitable for administering efficacious food compositions to canines. The kits comprise in separate containers in a single package or in separate containers in a virtual package, as appropriate for the kit component, one or more of (1) one or more ingredients suitable for consumption by a canine; (2) instructions for how to combine the ingredients and other kit components to produce a composition useful for providing a health benefit as described herein; (3) instructions for how to use the food composition to obtain such benefits; (4) one or more probiotics; (5) one or more inactivated probiotics; (6) one or more components of inactivated probiotics that promote health benefits similar to or the same as the probiotics, e.g., proteins, lipids, glycoproteins, and the like; (7) one or more prebiotics; (8) a device for preparing or combining the kit components to produce a composition suitable for administration to a canine; and (9) a device for administering the combined or prepared kit components to a canine. In one embodiment, the kit comprises one or more ingredients suitable for consumption by a canine. In another embodiment, the kit comprises instructions for how to combine the ingredients to produce a composition useful for obtaining a health benefit as described herein.

When the kit comprises a virtual package, the kit is limited to instructions in a virtual environment in combination with one or more physical kit components. The kit contains components in amounts sufficient for to obtain a health benefit as described herein. Typically, the kit components can be admixed just prior to consumption by a canine. The kits may contain the kit components in any of various combinations and/or mixtures. In one embodiment, the kit contains a container of food for consumption by a canine. The kit may contain additional items such as a device for mixing ingredients or a device for containing the admixture, e.g., a food bowl. In another embodiment, the food compositions can be mixed with additional nutritional supplements such as vitamins and minerals that promote good health in a canine. The components can be each provided in separate containers in a single package or in mixtures of various components in different packages. In some embodiments, the kits comprise one or more other ingredients suitable for consumption by a canine. In one aspect, such kits can comprise instructions describing how to combine the ingredients to form a food composition for consumption by the canine, generally by mixing the ingredients or by applying optional additives to the other ingredients, e.g., by sprinkling nutritional supplements on a food composition.

In a further aspect, a means for communicating information about or instructions for one or more of (1) using a food composition for obtaining one of the health benefits described herein; (2) contact information for consumers to use if they have a question regarding the methods and compositions described herein; and (3) nutritional information about the food composition can be provided. The communication means can be useful for instructing on the benefits of using the present methods or compositions and communicating the approved methods for administering food compositions to a canine. The means comprises one or more of a physical or electronic document, digital storage media, optical storage media, audio presentation, audiovisual display, or visual display containing the information or instructions. In one aspect, the means can be selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, methods for manufacturing a food composition comprising one or more other ingredients suitable for consumption by a canine, e.g., one or more of protein, fat, carbohydrate, fiber, vitamins, minerals, probiotics, prebiotics, and the like, can comprise admixing one or more of the ingredients suitable for consumption by a canine. The composition can be made according to any method suitable in the art.

In another aspect, a package useful for containing compositions described herein can comprise at least one material suitable for containing the food composition and a label affixed to the package containing a word or words, picture, design, acronym, slogan, phrase, or other device, or combination thereof that indicates that the contents of the package contains the food composition. In some embodiments, the label affixed to the package contains a word or words, picture, design, acronym, slogan, phrase, or other device, or combination thereof that indicates that the contents of the package contains the food composition with beneficial properties relating to a health benefit described herein. In one aspect, such device can comprise the words “efficacious weight loss diet,” or an equivalent or similar expression printed on the package. Any package configuration and packaging material suitable for containing the composition can be used herein, e.g., bag, box, bottle, can, pouch, and the like manufactured from paper, plastic, foil, metal, and the like. Generally, the package contains a food composition adapted for canine as appropriate for the label. In one embodiment, the package can be a can or pouch comprising a food composition described herein. In various embodiments, the package further comprises at least one window that permit the package contents to be viewed without opening the package. In some embodiments, the window can be a transparent portion of the packaging material. In others, the window can be a missing portion of the packaging material.

### EXAMPLES

The invention can be further illustrated by the following example, although it will be understood that this example is included merely for purposes of illustration and is not intended to limit the scope of the invention unless otherwise specifically indicated.

### Example 1—Dog Study

In this study, 30 overweight dogs were randomized into two groups with 15 dogs per group based on their baseline maintenance energy requirement (MER), body weight, % body fat, and genders. The dogs in both the control and test groups were fed 75% if their baseline MERs during the first 4 months of the weight loss study and then 60% of their baseline MERs during the last 2 months of the weight loss study. The body composition was determined with a DEXA machine. The diets are shown in Table 1.

TABLE 1

| Components | Test diet (wt %) | Control diet (wt %) |
|---|---|---|
| Moisture | 8.07 | 8.09 |
| Protein | 48.70 | 26.47 |
| Starch | 15.65 | 31.60 |
| Fat | 10.1 | 14.73 |
| Crude fiber | 5.00 | 11.40 |
| Total dietary fiber | 12.93 | 19.70 |
| Ash | 5.94 | 5.19 |
| n-3 PUFAs* | 1.2166 | 0.08902 |
| n-6 PUFAs** | 1.53587 | 1.62164 |
| Total Isoflavones (mg/kg) | 965.33 | 138.67 |

*Omega-3 Polyunsaturated fatty acids
**Omega-6 Polyunsaturated fatty acids

There was no significant difference in lean body mass between baseline and any of the three time points (2 months, 4 months, and 6 months) in dogs fed the test diet. On the contrary, dogs fed the control diet lost significant amount of lean body mass at all three time points compared with baseline as shown in Table 2.

TABLE 2

| | Lean Body Mass - Control diet | | | Lean Body Mass - Test diet | | |
|---|---|---|---|---|---|---|
| Time | Initial | Final | Difference | Initial | Final | Difference |
| Period | (kg) | (kg) | (kg) | (kg) | (kg) | (kg) |
| 2 months | 20.29 | 19.60 | −0.69 | 20.09 | 20.32 | 0.24 |
| 4 months | 20.29 | 19.75 | −0.54 | 20.09 | 20.08 | 0.05 |
| 6 months | 20.29 | 19.54 | −0.75 | 20.09 | 19.88 | −0.15 |

Dogs in both groups lost significant amount of body fat compared with baseline. However, dogs fed the test diet lost more body fat than the control dogs (5.93 kg vs 4.98 kg) at the end of the 6-month weight loss study as shown in Table 3.

TABLE 3

| | Body Fat - Control diet | | | Body Fat - Test diet | | |
|---|---|---|---|---|---|---|
| Time | Initial | Final | Difference | Initial | Final | Difference |
| Period | (kg) | (kg) | (kg) | (kg) | (kg) | (kg) |
| 2 months | 13.35 | 12.00 | −1.35 | 13.38 | 11.31 | −2.07 |
| 4 months | 13.35 | 10.47 | −2.88 | 13.38 | 9.60 | −3.68 |
| 6 months | 13.35 | 8.37 | −4.98 | 13.38 | 7.35 | −5.93 |

Several markers associated with muscle growth/breakdown were analyzed. Surprisingly, 2 markers were found that distinguished the present test diet from traditional diets such as the control diet. The results are shown in Table 4.

TABLE 4

% Changes over baseline

| Amino Acid Markers | Control Diet | Test Diet |
|---|---|---|
| Histidine | No change | No Change |
| Leucine | No change | +11% |
| Valine | No change | +9% |
| Threonine | −8% | −21% |
| Methionine | −8% | −21% |
| 3-methylhistine | No change | −42% |
| 1-methylhistidine | +12% | −29% |

As shown in Table 4, 3-methylhistidine and 1-methylhistidine were shown to significantly differentiate the test diet from the control diet. As such, 3-methylhistidine and 1-methylhistidine can be used as diagnostic markers for determining the efficacy of weight loss diets. The use of these markers can aid a veterinarian in determining whether a weight loss diet is a healthy weight loss diet, e.g., preserves lean body mass, or an unhealthy weight loss diet, e.g., loss of significant lean body mass without the need of expensive or unavailable DEXA (dual-energy X-ray absorptiometry) or QMR (quantitative magnetic resonance) scanners.

What is claimed is:

1. A method of determining the efficacy of a weight loss diet for a canine, the method comprising:
analyzing a first serum or plasma sample from the canine for a biomarker selected from the group consisting of 3-methylhistidine, 1-methylhistidine, and a combination thereof;
determining the amount of the biomarker in the first serum or plasma sample;
analyzing a second serum or plasma sample for the biomarker after the canine has been administered the weight loss diet;
determining the amount of the biomarker in the second serum or plasma sample; and
determining the weight loss diet is efficacious if the amount of the biomarker in the second serum or plasma sample has decreased from the amount of the biomarker in the first serum sample or determining the weight loss diet is inefficacious if the amount of the biomarker in the second serum or plasma sample has not changed or increased from the amount of the biomarker in the first serum or plasma sample.

2. The method of claim 1, wherein the weight loss diet has been administered to the canine on a regular basis.

3. The method of claim 1, wherein the weight loss diet has been administered to the canine for at least 1 month.

4. The method of claim 1, wherein the weight loss comprises a complete and balanced pet food.

5. The method of claim 1, further comprising administering the weight loss diet to the canine.

6. The method of claim 1, wherein, when the weight loss diet is efficacious, the weight loss diet provides a health benefit selected from the group consisting of: preservation of lean body mass, minimization of lean body mass during weight loss, reduced body fat, reduced weight, reduced weight gain, reduced insulin resistance, decreased risk of diabetes, decreased risk of prediabetes, lower cholesterol, lower glucose, lower triglycerides, lower insulin, lower leptin, improved insulin sensitivity, prevention of prediabetes, delaying onset of prediabetes, treatment of prediabetes, prevention of diabetes, delaying onset of diabetes, treatment of diabetes, prevention of insulin resistance, delaying onset of insulin resistance, treatment of insulin resistance, prevention of overweight or obesity, delaying onset of overweight or obesity, treatment of overweight or obesity, promoting metabolic health, promoting better blood glucose management, lowering chronic inflammation and proinflammatory cytokines, improving voluntary daytime activity, reducing restlessness at daytime and nighttime, increasing satiety, and combinations thereof.

7. The method of claim 1, wherein, when the weight loss diet is inefficacious, advising a different weight loss diet for the canine.

8. A method of providing an efficacious weight loss diet for a canine, the method comprising:
analyzing a first serum or plasma sample from the canine for a biomarker selected from the group consisting of 3-methylhistidine, 1-methylhistidine, and a combination thereof;
determining the amount of the biomarker in the first serum or plasma sample;
analyzing a second serum or plasma sample for the biomarker after the canine has been administered the weight loss diet;
determining the amount of the biomarker in the second serum or plasma sample;
determining the weight loss diet is efficacious if the amount of the biomarker in the second serum or plasma sample has decreased from the amount of the biomarker in the first serum or plasma sample or determining the weight loss diet is inefficacious if the amount of the biomarker in the second serum or plasma sample has not changed or increased from the amount of the biomarker in the first serum or plasma sample; and
wherein, when the weight loss diet is inefficacious, providing an efficacious weight loss diet to the canine that comprises protein, fat, carbohydrates, omega-3 fatty acids, and isoflavones; wherein the protein to carbohydrate is in a ratio ranging from 4.5:1 to 2.5:1 by weight as fed.

9. The method of claim 8, wherein the omega-3 fatty acids are selected from the group consisting of eicosapentaenoic acid (EPA), docosahexaenoic acid (DHA), alpha-linolenic acid (ALA) and mixtures thereof; and wherein the omega-3 fatty acids are present in an amount from about 0.1% to about 5% by weight of the pet food composition.

10. The method of claim 8, wherein the isoflavones are selected from the group consisting of daidzein, 6-O-malonyl daidzein, 6-O-acetyl daidzein, genistein, 6-O-malonyl genistein, 6-O-acetyl genistein, glycitein, 6-O-malonyl glycitein, 6-O-acetyl glycitein, biochanin A, formononetin, or mixtures thereof.

11. The method of claim 8, wherein the isoflavones are present in an amount from about 300 mg to 1,500 mg per kilogram of the pet food composition.

12. The method of claim 8, wherein the protein is present in an amount from about 30% to about 60% by weight of the pet food composition and the carbohydrates are present in an amount from about 5% to about 25% by weight of the pet food composition.

13. The method of claim 8, wherein the protein to carbohydrate is in a ratio ranging from 4.25:1 to 3.75:1.

14. The method of claim 8, wherein the efficacious weight loss diet is administered to the canine on a regular basis.

15. The method of claim 8, wherein the efficacious weight loss diet is administered to the canine for at least 1 month.
